(19) United States
(12) Patent Application Publication
(10) Pub. No.: US 2004/0049351 A1
(43) Pub. Date: Mar. 11, 2004

(54) Immunosorbent assay in microarray format

(76) Inventors: Robert S. Matson, Orange, CA (US); Jang B. Rampal, Yorba Linda, CA (US)

Correspondence Address: PATENT LEGAL DEPARTMENT/A-42-C, BECKMAN COULTER, INC., 4300 N. HARBOR BOULEVARD, BOX 3100, FULLERTON, CA, 92834-3100, US

(21) Appl. No.: 10/230,028

(22) Filed: Aug. 28, 2002

(51) Int. Cl. C12Q 1/68; G01N 33/53; G06F 19/00; G01N 33/48; G01N 33/50

(52) U.S. Cl. 702/19; 435/6; 435/7.1

(57) ABSTRACT

A multiplexed immunosorbent assay can be performed in a microarray format on a plate. Capture molecules corresponding to the specific analytes are printed onto the bottom of the wells of chemically activated plates. The conditions are optimized for printing in terms of capture molecule spot density (mass and uniformity), coupling conditions, and blocking conditions. Samples containing analytes to be detected are delivered to the wells, allowed to incubate for a specific time after which unbound sample is removed by rinsing. Detection secondary capture molecules are pre-mixed and delivered to each well. Following incubation and rinse, signal generation reagents are added and the signals are detected.

### FIELD OF THE INVENTION

The present invention relates to a multiplexed immunosorbent assay in a microarray format. The invention also relates to the use of a multi-well plate in automated microarray immunoassays.

### BACKGROUND OF THE INVENTION

Reactions between biological molecules exhibit an extremely high degree of specificity, which provides a living cell with the ability to carry out thousands of chemical reaction simultaneously in the same vessel. Generally, this specificity arises from the fit between two molecules having very complex surface topologies. Examples of these are an antibody binding a molecule displaying an antigen on its surface because the antibody contains a pocket whose shape is the complement of a protruding area on an antigen. Tests that detect the presence of DNA or RNA that is complementary to a known DNA or RNA chain are based upon the sequences in the chains such that an A in one chain is always matched to a T in the other chain, and a C in one chain is always matched to a G in the other chain, binding the two chains together by electrostatic forces.

Systems for medical diagnosis often involve a bank of tests in which each test involves the measurement of the binding of one mobile component to a corresponding immobilized component. To provide inexpensive test kits, systems involving a matrix of immobilized spots has been suggested wherein each spot includes the immobilized component of a two component test such as described above. The fluid to be tested is typically brought into contact with the matrix. After rinsing away unbound sample, the presence of the analyte within the sample is determined by its location within the matrix using labeled markers to determine the presence of the analyte.

### SUMMARY OF THE INVENTION

It is an object of the present invention to overcome deficiencies in the prior art.

It is another object of the present invention to provide an immunoassy using both analyte specific binding agents and control specific binding agents, whereby each individual assay generates a standard curve.

It is another object of the present invention to provide a micro-immunosorbent assay comprising multiple wells wherein n=the number of elements in a well, in a plate such that up to 9633 n, 38433 n, or 153633 n microassays can be conducted at once.

It is another object of the present invention to conduct micro-immunosorbent assays in an automated fashion using a robotic system.

It is a further object of the present invention to provide a micro-immunosorbent assay system in which at least one well, or selective wells, contains a plurality of antibodies or other capture molecules along with controls.

It is another object of the present invention to provide a micro-immunosorbent assay in which each analyte assay is treated independently of other assays.

It is another object of the present invention to provide a micro-immunosorbent assay system in which not all wells contain controls.

It is still another object of the present invention to create arrays of affinity-based receptors or molecular recognition elements which permit self-assembly of user defined analyte specific capture ligands.

According to the present invention, a multiplexed immunosorbent assay is performed in a microarray format on a plate or other substrate. Capture molecules corresponding to the specific analytes, and capture molecules corresponding to controls, are printed onto the surface of a substrate, such as a chemically activated multiwell plate. The conditions are optimized for printing in terms of capture molecule spot density (mass and uniformity), coupling conditions, and blocking conditions. Samples containing analytes to be detected and controls are delivered to the individual spots, allowed to incubate for a specific time (e.g., one hour), and then unbound sample is removed by rinsing. Detection secondary capture molecules are pre-mixed and delivered to each well. Following incubation and rinse, signal generation reagents are added and the signals are detected.

Analyte concentration within a single well is made in relation to controls assayed within the same well. Alternatively, analyte can be assayed in one well and control assayed in a different well.

The process of the present invention provides a process wherein the delivery, capture, and assaying of multiple analytes and controls can be effected randomly, sequentially, or in parallel. This makes it possible to use the process of the present invention in automated assays. Certain of these automated assays may require simultaneous delivery of the reagents to all of the wells of a multiwell plate, while other assays may require delivery of reagents to the wells sequentially or only to selected wells.

For quality control, it is at times desirable to perform assays by random selection of wells. This process comprises contacting the bottom surface of wells in a multi-well plate comprised of activated surfaces with an array of molecular recognition capture elements corresponding to specific analytes and controls. Analytes and controls are then delivered randomly, sequentially, or in parallel to the molecular recognition capture elements in the wells; the wells can contain both analytes and controls, or only analytes or only controls. The analytes and controls are then incubated to form complexes. The plate is rinsed to remove unbound analytes and controls, after which a solution of the same or different molecular recognition signal reporterss that bind to analytes and controls are delivered to each well randomly, sequentially, or in parallel. The molecular recognition signal reporterss are then incubated, and the plates are rinsed to remove unbound molecular recognition signal reporters. Signal generation reagents, which may be the same or different, are added, and signals, which may be the same or different, generated from bound analytes and controls are detected.

One advantage of the system of the present invention is that multiple antigens can be simultaneously analyzed without the cross-reactivity associated with capture antibody or secondary antibody reactions. Another advantage of the system of the present invention is that the system can be used with unmodified biological molecules which are immobilized on activated substrates, particularly substrates which have been activated with acyl fluoride. Methods for immobilizing these biological molecules are described in detail in Matson et al., U.S. Pat. No. 6,268,141, the entire contents of which are hereby incorporated by reference.

Each spot or well has its own assay. That is, each spot or well assay is treated independently from the other spot or well assays, thus minimizing cross-reactivity. It is also possible to link assays among multiple wells, such as in serial dilution of analyte to measure the limits of detection and dynamic range.

Proteins, for example, are assayed according to the present invention comprising the steps of:

a. contacting wells of activated substrates with capture monoclonal antibodies corresponding to specific proteins and to controls;

b. delivering antigens to the proteins and to the controls to the wells either as antigen and control in each well, or antigens and control in separate wells, and incubating the antigens and controls with the capture monoclonal antibodies to form complexes;

c. rinsing to remove unbound antigens;

d. delivering secondary antibodies to each well;

e. incubating the secondary antibodies with the contents of the wells and rinsing to remove unbound secondary antibodies;

f. adding signal generation reagents; and

g. detecting the signal generated.

Preferably, the assays are automated and are performed by computer-controlled robots, which deliver samples, controls, and to a multi-well plate. Kits for use in manual or automated assays include optimized labeling and detection or reagents, wash buffers, etc. for a given assay.

According to another aspect of the present invention, arrays of molecular recognition elements are immobilized in the bottom of plate wells in a pre-defined order such that accurate registration along both the x and y axes is known. For example, the recognition element is an antibody that recognizes a specific hapten molecule. In its simplest form, the following reagents are needed to create a universal assay in its simplest form:

1. a library of anti-hapten antibodies

2. a library of corresponding haptenated capture antibodies.

This technique can be used with a variety of multiplex formats, such as bead based or fiber optic-based arrays. This technique provides flexibility and cost savings in the manufacture of plates and associated reagents. New assays can be more rapidly developed using a standardized plate format.

For purposes of the present invention, the analytes are described as antigens that are recognized by antibodies, their analogs or mimics. However, if the antibody used in the assay can recognize a hapten, a drug or other small organic molecule, nucleic acid, phosphoplipid, etc., then these can also be analyzed by the method of the present invention, and are included in the analytes that can be detected by this invention.

For purposes of the present invention, controls are defined as members of a binding pair which is not a member of the binding pair which includes the analyte of interest. The controls can be antigens that are recognized by antibodies, their analogs or mimics.

### BRIEF DESCRIPTION OF THE DRAWINGS

FIG. 1 illustrates a multiplexed microarray ELISA.

FIG. 2 shows a 4×4 printing of anti-IL-4 monoclonal antibody in a plate.

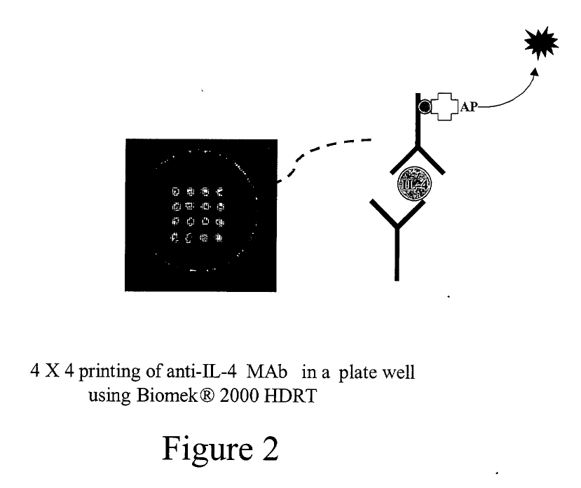

FIG. 3 shows molecular recognition elements immobilized on a solid phase in position to recognize a particular hapten molecule.

FIG. 4 illustrates a multiple array plate well including molecular recognition elements immobilized therein.

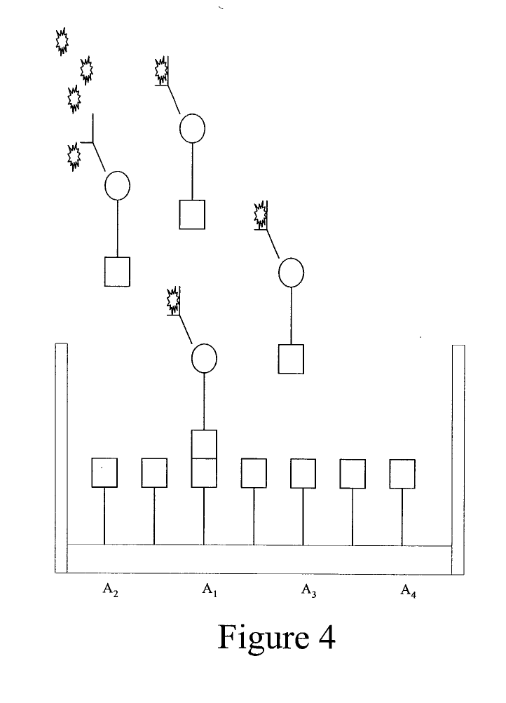

### DETAILED DESCRIPTION OF THE INVENTION

While the assays of the present invention can be conducted on any suitable substrate, the preferred substrate is a multiple array microplate, the subject of U.S. application Ser. No. 09/675,020, the entire contents of which are hereby incorporated by reference. This multiple array microplate is a device comprised of multiple wells, wherein the wells are discrete areas separated by barriers such as walls, hydrophobic patches, troughs, gaskets, pedestals, or the like, that restrict fluid cross-flow between the discrete areas. An array of immobilized elements may be formed within each well. For purposes of the present invention, an element is defined as a discrete, physical location for biorecognition materials. The number of elements in an array may range from about 1 to about 1536 or more, and preferably from about 16 to about 400. The size of the arrays may be the same or different in different wells. The elements in each array may contain the same or different biorecognition materials.

A Biorecognition materials@ generally refers to materials that interact with target materials in the sample to recognize the targets, as well as with controls to recognize the controls. Biorecognition materials, also known as immunoreactants, that may be immobilized on the plate include biomolecules such as DNA; proteins; cells and cellular components such as membrane receptors, biomolecule recognition sites, suborganelles, and other structural features. Of particular importance are proteins, including antigens, enzymes, receptors, or small compounds such as peptides.

Biorecognition agents for the purposes of the present invention are defined as compound such as a protein (i.e., an amino acid sequence containing more than 50 amino acids) or peptide (i.e., an amino acid sequence comprising fewer than 50 amino acids) or other molecules which bind to the analyte. Typically the immunoreactant is a monoclonal or polyclonal antibody to the analyte or a portion thereof, such as a Fab=fragment, which specifically binds to the analyte. However, as one skilled in the art can readily appreciate, the formation of a specific conjugate comparable to the binding of an antibody to an antigen may be achieved through the use of another specific protein- or peptide-based binding system, such as a receptor protein or fragment thereof and a ligand therefore, which would not generally be considered to involved in immunochemical conjugation. Further, analogs and variants or mimics of various immunoreactants, such as those generated using recombinant DNA techniques, which specifically bind to the target analyte, are contemplated to be within the scope of the present invention.

The biorecognition materials are attached to the surface of the well by covalent bonding, non-covalent bonding, or any other suitable means, such as affinity interaction with biorecognition molecules attached to the site. For example, a covalent attachment using acyl fluoride chemistry may preferably be used. Cells or cellular components may be attached to the wells via cell surface constituents such as proteins, carbohydrates, glycoproteins or other biomolecules or linkers. The elements within each well or within each plate may be labeled with the same or different labels. The samples to be added to the wells may also be labeled with one or more labels. The sample may also be a mixture of different biological samples, each being labeled or unlabeled.

For purposes of the present invention, a condition is sufficient if the agent can bind to the target molecule to form a complex. This condition may vary, depending upon the type of molecules and the type of bindings. One skilled in the art can readily determine suitable conditions for binding in view of the teachings of the present invention.

Either the target molecules or the agents can be labeled with a reporter molecule. Examples of reporter molecules include, but are not limited to, dyes, chemiluminescent compounds, enzymes, fluorescent compounds, metal complexes, magnetic particles, biotin, haptens, radio frequency transmitters, and radioluminescent compounds. One skilled in the art can readily determine the optimum type of reporter molecule to be used for each target molecule.

The multiple array plate is preferably formed of plastic material that can be surface-treated for immobilization of the biorecognition material. Preferred materials include thermoplastics such as polypropylene, polyethylene, and/or their copolymer blends, although other materials and combinations thereof such as polymeric foams, gels, glasses or ceramics can be used as long as they can be formed into wells or barriers and surface-activated. Rather than directly treating the surface of the plastic, an activated insert may be placed into the well, such as a disk, screen, foam, or filter material. Alternatively, an activated coating may be adsorbed in the well. The bulk plastics are preferably chemically inert and characterized by low nonspecific adsorption of biomolecules and other biorecognition materials, and low intrinsic or auto-fluorescence. It is preferred that the plastic material be sufficiently transparent and of good optical quality to allow light transmission and detection through the bottom of the well (transillumination reading). The signal can also be detected from excitation by a reflected light (epiillumination reading).

The plate may have any number of wells and any well pattern and geometry as needed for specific applications. For example, the entire tray may be one well, or a large number, such as 2,000 or more small wells, may be molded therein. In a preferred embodiment, the plate has an 8×12 array of 96 wells, each well being 6 mm in diameter and about 1 to 4 mm in depth, and the distance between wells is 9 mm center-to-center.

In another embodiment, a plate having enhanced liquid handling ability is formed with a flat array formation area surrounded by a peripheral depression. An array of microarrays is printed on the flat array formation area, and the samples are added to the area and subsequently removed from the peripheral depression with a pipette tip. This geometry provides a larger printing surface, enhances liquid handling ability by diverting the fluid away from the central flat area, and improves array imaging.

While the frame is formed of a rigid material for support, the tray is formed of a flexible material. Preferably, the tray is formed of a thermally formable polymer sheet, by vacuum forming or injection molding. In a preferred embodiment, the tray is formed of polypropylene and has a thickness of about 0.1 to 100 mils, preferably about 1 to 10 mils, a flexural modulus (ASTM D790) of about 170-220 Ksi, a Shore D hardness (ASTM D 2240) of about 65-80, and a deflection temperature at 66 Psi of about l00-200ΕF.

During the microarray assay process, the multiple array plate may be used in conjunction with a fixture apparatus such that when the plate is placed on the fixture, the bottom of each well is seated flat on the top surface of the fixture. The fixture apparatus is provided with various functions for different processing needs, such as maintaining a flat well bottom surface during microarray printing and data reading processes, controlling the well temperature and/or micromixing the well contents during heating, etc.

In another embodiment, the top surface of the fixture is formed with a plurality of depressions having a shape complementary to the shape of the tray of the plate, so that when the plate is mounted on the fixture, the wells of the plate sit within the depressions and the bottom of each well is flat against the bottom of the depression.

In an alternative embodiment, the top surface of the fixture is flat and the bottom of the wells are placed against the flat surface when the plate is mounted on the fixture.

In still another embodiment, the frame of the plate is shaped to be mounted on the fixture so that the tray rests on the top surface of the fixture. The fixture in turn is mounted on various processing apparatuses such as printing machine, incubator, etc. as described later.

The fixture has an interior chamber connectable to a vacuum source via channels, and a plurality of orifices located on the top surface and connected to the interior number. The orifices are located within the depressions or at locations corresponding to the bottom of the wells. When a vacuum is drawn in the interior chamber, the vacuum is communicated via the orifices to create a negative pressure to hold the bottom of the wells firmly against the top surface of the fixture. As a result, even though the tray is formed of a flexible material, the bottom portions of the wells maintain a high precision flatness to facilitate high-resolution printing and reading of the microarrays. The flatness of the well bottom is generally determined by the flatness of the depressions or the top surface of the fixture corresponding to the bottom of the wells. A high degrees of flatness of less than 0.0001-inch variation across the tray may be obtained.

Temperature control abilities may be provided to the vacuum fixture by providing a plurality of channels in the fixture to pass a temperature-controlled fluid. Alternatively, temperature control may be achieved by using a resistance heater, or by using a layer of solid state thermoelectric material such as a Peltier type material disposed between the surface of the fixture and the tray to provide cooling. In a temperature-control fixture, the orifices function to remove air from between the bottom of the well and the surface of the fixture to reduce the thermal resistance between the fixture and the tray. This ensures uniform temperature control for the wells.

In addition, the vacuum fixture may be provided with a micromixing capability by connecting the vacuum chamber to a peristaltic pump which generates alternating positive and negative pressures. The alternating pressures are optionally communicated by the orifice to the space between the surface of the fixture and the bottom of the well, causing the flexible bottom portion of the well to be alternately pushed up and pulled down. This creates a micromixing effect to uniformly mix the solution held in the well.

The physical dimensions and properties of the multiple array plate and the fixture apparatus may be selected so that they are adapted for working with existing or future microarray assay devices. For example, the plate may be designed to conform to operation on the Biomek series Workstation Platforms (Beckman Coulter, Inc., Fullerton, Calif.) or similar robotic liquid handlers in order to automate the assay completely. The outside linear dimensions of the multiple array plate may be made to conform to a standard microtiter plate footprint with a rigid frame, conventionally used in automated assays in which the microplate is moved from one location to another on the workstation and/or its peripheral networked devices. In addition, the wells of the multiple array plate are sufficiently shallow (such as less than 4 mm deep) with a side draft that allows for within well reading of individual wells by a CCD camera or other detector system. Specific properties of the plastic tray material such as thickness, tensile strength, elongation and elasticity modulus shear strength, flatness, heat capacity, solvent and water adsorption properties, well shape, etc. are selected in order for the multiple array plate to deliver optimal performance when used with particular fixture apparatus and assay devices. For example, if the mixing feature is to be used, the material is selected so that the multiple array plate is capable of flexing up and down by the action of a peristaltic pump.

According to another embodiment of the present invention, a microwell formed using a die cut gasket is sealed to an activated plastic substrate containing a microarray of biorecognition materials. Optionally, the plastic substrate is attached to a stiff support plate to provide strength. The gasket, the plastic substrate and the support plate are held together, such as by using adhesive, sonic welding, compression fitting or vacuum forming, to form a shallow well microplate. This multiple array plate may also be used with a vacuum fixture device.

The multiple array plate may also be provided with a lid, such as a vacuum clamped lid, to control the micro environment of the wells for cell culturing or sensitive assay development, such as to reduce contamination, retard evaporation, prevent condensation, and/or provide temperature control. Since assays are typically conducted at either 25ΕC or 37ΕC, a lid is preferably used when the assay is conducted at the higher temperature. Alternatively, tape or sealing film may be used in place of a lid. The lid may also be designed to allow for cell culturing, by providing both temperature control and ports for gas exchange over the liquid in the well to maintain partial gas pressure and pH control needed for cell growth.

According to another embodiment of the present invention, a multiple array plate is formed of a plurality of flexible strips each comprising a plurality of wells arranged in one or more rows. The strips may be formed of a flexible plastic material by molding or vacuum forming, and they may be separately formed or sectioned into strips from a previously formed plate. Each flexible well strip is press-fitted into a rigid plastic hanger, which is in turn mounted on a rigid plastic frame.

According to yet another embodiment a multiple array plate may be formed of individual molded or vacuum-formed plastic wells fitted into a rigid plastic hanger comprising a ring and crossbars. A series of hangers is linked together and to a frame of rigid plastic. The plate according to these embodiments allows a single well or a group of wells on a plate to be handled separately and increases the versatility of the device.

The multiple array plates according to embodiments of the present invention may be used as consumables on specialized workstations such as the Biomek line of liquid handling robots. The plates may be pre-printed with arrays of probes in the wells or unprinted.

The assay system of the present invention makes it possible to completely automate the assay process and increase reliability throughout. Standard protocols can be written for the automatic handling process, and variations inherent in manual processing are removed. All members of the array can be analyzed at one time, or selected members of the array can be analyzed at separate times.

For the purpose of the present invention, it is not crucial which particular method is used to carry out the step of contacting the substrate with the capture molecule and control. In accordance with embodiments of the present invention, the contacting step may be carried out by jet printing, solid or open capillary device contact printing, microfluidic channel printing, silk screening, or printing using devices based upon electrochemical or electromagnetic forces. For example, thermal inkjet printing techniques using commercially available jet printers and piezoelectric microjet printing techniques, as described in U.S. Pat. No. 4,877,745, the entire contents of which are hereby incorporated by reference, may be used to spot proteins to the acylated substrates. A Biomek High Density Replicating Tool (HDRT) (Beckman Coulter, Calif.) may also be used for automatic gridding.

The capture molecules and controls may be provided in the wells of the multiple array plate by first providing a solution of the capture molecule and control, placing an aliquot of this solution in the well, and air-drying the substrate to directly adsorb the capture molecule and control on the surface of the well.

The concentration of capture molecules and controls contained in aqueous solutions may vary, depending upon the size of the molecules, the structure of the molecules, and other factors that may influence the solubility of the molecules. Preferably, the amount of capture molecule and control applied to the substrate ranges from about 1 zeptomole (10⁻²¹ moles) to about 1 micromole (10⁶ moles). The size of the aliquot is not crucial, so long as it provides sufficient amount of the capture molecule and control for assay. Consequently, the size of aliquots applied to the treated substrate may vary, depending upon the concentration of the capture molecule control in the solution and the assay need.

In accordance with the present invention, the air-drying step is conducted for a period of time sufficient to allow adsorption of the capture molecule/control solution. The length of time required for air-drying depends on the volume of the aliquots applied to the substrate, room temperature, and humidity. For micro- and nanoliter aliquots the air-drying step may take from about 5 minutes to about 60 minutes.

To form arrays of the proteins, with each capture molecule and control located at a site-specific location, including grids and 1×n arrays of immobilized capture molecule and controls, a preselected site on the surface of the substrate is exposed to a solution of the desired capture molecule and control. This can be accomplished manually by applying an aliquot of appropriate solution to a preselected location on the substrate. Alternatively, thermal inkject printing techniques using commercially available inkjet printers and piezoelectric microjet printing techniques can be use used to spot selected substrate surface sites with selected capture molecules and controls.

A wide variety of array formats can be used in accordance with the present invention. One particularly useful format is a linear array of protein probes, generally referred to in the art as a dipstick. Another suitable format comprises a two-dimensional pattern of discrete spots. Of course, one skilled in the art can appreciate that other array formats are equally suitable for use in the present invention.

The assay of the present invention is not limited to use with a multiple array plate. Any suitable substrate or multi-well plate can be used that would allow for random access and sequential or parallel processing of the samples.

In the assay of the present invention, the molecular recognition capture elements, analytes, and controls can be proteins, peptides, haptamers, antibodies, antigens, enzymes, haptens, and receptors, as well as corresponding analogs or mimics. For purposes of the present invention, Aanalogs@ and Amimics@ are compounds that function in substantially the same way immunologically as the compounds of which they are analogs or mimics. The labeled forms of the molecular recognition capture elements, analytes, and controls and their analogs or mimics can include enzymes, enzyme substrates, mass labels, dyes, metals, radiolabels, or hapten conjugates or complexes of the reporters.

In one embodiment of the present invention, the array of molecular recognition capture elements are antibodies, and the controls are antigens, their analogs, and mimics. These antigens may be interleukins, cytokines, chemokines, growth factors, hormones, or transcription factors.

Molecular recognition capture elements, analytes, and controls may be proteins associated with cytokine signaling pathways, MAP kinases, Akt signaling pathways, PKC pathways, apoptosis and caspase signaling pathways, and proteins involved in cell cycle and translational control. Examples would include but are not limited to:

cytokine signaling pathways: Stat1, Stat3, Stat5, Stat6, Tyk2, Smad1, IkB-a,

MAP kinase: MAPK, Erk1/2, p44/42 MAP kinase, Raf, MEK-1/2, MEK 1 inhibitor, MEK-1/2 inhibitor, p90RSK, RSK3, MSK1,

Akt signaling pathway: Akt, GSK-3 (glycogen synthase kinase), Bad, pEBG, eNOS, FKHR, AFX, PTEN, PI3 kinase inhibitor

PKC pathway: PKC, PKCα/β_{II}, PKCδ, PKD/PKCμ, PKCθ, PKCζ/λ

Apoptosis/Caspase Signaling pathways: Cleavage-specific panels:Caspases (cleaved vs. uncleaved): caspase 3, 6, 7, 8, 9, 10, PARP, Lamin A (substrate for caspase 6), a-Fodrin, DAP1, 3, 5, BID, XIAP,

Translational control/WNT pathways:Antibodies directed against phosphorylated and unphosphorylated versions of Mnk1, 4E-BP1, p70 S6 Kinase, S6 Ribosomal Protein, eEF2, eIF2α, FRAP/mTOR inhibitor, β-catenin

Cell cycle control targets: p53, cdc2, cdc25, Rb Alternatively, the molecular recognition capture elements, analytes, and controls can be proteins that participate or are associated with phosphorylation, dephosphorylation, glycosylation, deglycosylation, acylation and deacylation, methylation and demethylation of molecules, for example, but not limited to: cytokine and other mitogen activation of various protein kinase cascades; Cdc25 (dephosphorylation of MPK);Cyclin phosphorylation by cyclin activating kinase (CAK); MPF phosphorylation of histone 1; TGF-β mediated phosphorylation cascade activation of Smad2; histone acylation by HAT (histone acyltransferase); Calcium/calmodulin dependent protein kinase (CaMK-II/IV) mediated protein phoshorylations, etc.

In another embodiment of the present invention, the molecular recognition capture elements are different haptenated proteins conjugated with different capture antibodies, haptamers, their analogs or mimics. The array can be formed by self-assembly of the elements onto corresponding anti-hapten antibodies, haptamers, their analogs or mimics arrayed on the bottom surface of wells at defined locations.

The microimmunosorbent assay of the present invention lends itself well to being conducted by a computer-controlled, automated device. A preferred automated device is a robotic device, such as the Biomek produced by Beckman Coulter, Inc. For example, a kit according to the present invention can comprise a panel of antibodies to cytokine signaling pathway proteins. This kit would include molecular recognition elements (anti-cytokines), analyte (serum sample containing cytokines), control (purified cytokines of known concentration), molecular recognition signal reporters (2^{nd} antibody, e.g ., biotinylated anti-cytokines), signal generation reagents (streptavidin-conjugate, e.g., streptavidin-FITC), and appropriate buffers, etc.

The present invention also provides for a universal array comprising:

a library of anti-hapten antibodies; and

a library of corresponding haptenated capture antibodies.

The haptenated capture antibody library can comprise a variety of different haptenated proteins conjugated with different capture antibodies, haptamers, their analogs or mimics. The array can be formed by self-assembly of the haptenated protein conjugates onto corresponding anti-hapten antibodies, haptamers, their analogs or mimics which are located at known locations on the bottom of wells of a multi-well plate. As with the microimmunsorbent assay, the multi-well plate can be a multiple array plate.

In summary, the microarray assay system of the present invention provides significantly higher throughput as compared to conventional systems using treated glass slides as array substrate. While the glass slides are a commodity and meet the flatness criteria, it is tedious and time-consuming to process, store and catalog hundreds and thousands of slides generated each month in many molecular biology labs. Also, each glass slide has a glass cover slip that must be applied and removed by hand. The use of a multiple array microwell plate, on the other hand, means that many samples can be processed with one plate. The plates can be bar code labeled for tracking. The microarray assay system also minimizes variations in critical parameters in the printing, hybridization or binding assay and imaging of microarrays, thereby improving repeatability of the assay. Performance metrics obtained from the microarray assay system of the present invention is equivalent to or better than existing immunoassays. The invention may be used in gene expression, SNPs, immunoassays, cell assays, etc.

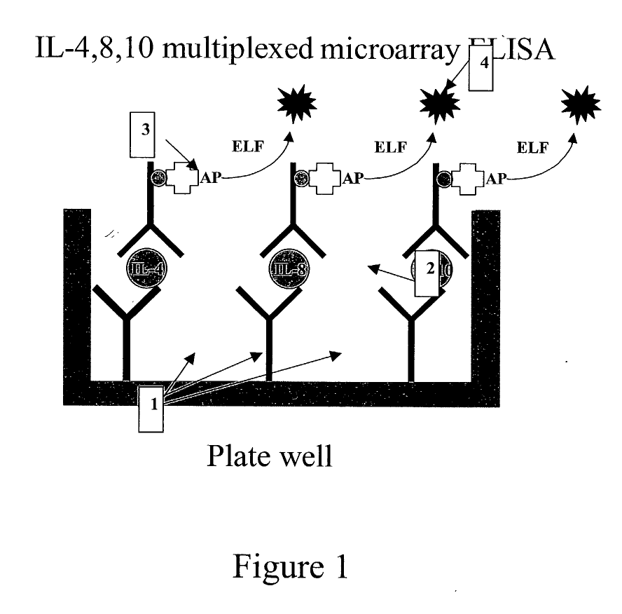

As shown in FIG. 1, capture monoclonal antibodies 1 corresponding to specific antigens 2, such as interleukins, were printed using Biomek 2000 HRDT pins onto the bottom of the wells of acyl fluoride activated microwell plates. The conditions were optimized for printing in terms of capture MAb spot density (mass and uniformity), coupling conditions, and blocking conditions. Antigens were delivered to the wells and allowed to incubate for a specified time, in this case, one hour. Unbound antigen was then removed by rinsing. Detection secondary antibodies 3 as controls were pre-mixed and delivered to each well. Following incubation and rinse, signal generation reagents 4, such as ELF (enzyme labeled fluorescent substrate, Molecular Probes, Inc., which is a substrate for alkaline phosphatase) were added and the signal was detected using a charge-couple device (CCD) camera system. The feasibility of this assay system has been demonstrated, and showed that multiple antigens could be simultaneously analyzed without cross-reactivity associated with capture antibody or secondary antibody interactions. As described above, the detection of interleukin antigens spiked into cell culture media containing 10% fetal calf serum was specific and sensitive. The sensitivity and linear dynamic range was comparable to that of a leading ELISA kit.

A multiplexed micro-ELISA according to the present invention shows the following specificity:

TABLE I
Microwell plate multiplexed micro-ESISA: cross reactivity
A plate multiplexed micro-ELISA: cross-reactivity
Antigen Mixes

| | | IL-4, | | | | | | |
|---|---|---|---|---|---|---|---|---|
| | | 8, 10 | IL-4, 8 | IL-4, 10 | IL-8, 10 | IL-4, 8, 10 | | |
| LL | 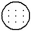 | 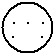 | 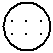 | 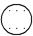 | 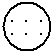 | 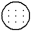 | 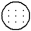 | LL |
| | | 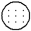 | 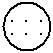 | 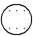 | 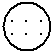 | 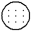 | | |
| | | 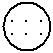 | 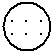 | 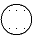 | 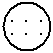 | 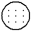 | | |
| | | 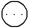 | 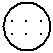 | 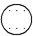 | 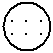 | 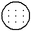 | | |
| | | 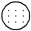 | 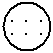 | 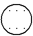 | 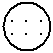 | 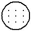 | | |
| | | 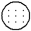 | 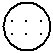 | 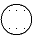 | 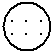 | 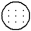 | | |
| | | 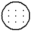 | 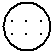 | 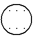 | 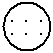 | 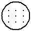 | | |
| LL | 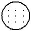 | 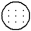 | 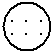 | 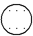 | 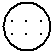 | 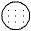 | 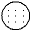 | LL |

A plate 43 ELF, 1 hr
Antigen Mixes
IL-4 = 500 pg/well
IL-8 = 50 pg/well
IL-10 = 5000 pg/well
LL = landing lights, Hu IgG
DetectionAb Mixtures:
anti-IL-4, 8, 10
1.100
1:1000 (column 2)

In the example shown in Table 1, antigens were applied in mixtures at high input concentrations to determine the extent of cross-reactivity. Under the conditions of the present invention, no evidence of cross-reactivity was observed.

Table 2 shows detection sensitivity of a plate multiplexed micro-ELISA according to the present invention.

TABLE II
Microwell plate multiplexed micro-ELISA: detection sensitivity
A plate multiplexed micro-ELISA: detection sensitivity

| | | IL-4 | | IL-8 | | IL-10 | | IL-4 | | IL-8 | | IL-10 | |
|---|---|---|---|---|---|---|---|---|---|---|---|---|---|
| LL | 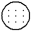 | 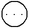 | 500 | 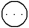 | 50.0 | 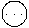 | 5000 | 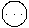 | 500 | 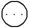 | 50.0 | 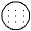 | LL |
| | | 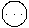 | 250 | 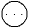 | 25.0 | 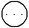 | 2500 | 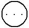 | 250 | 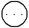 | 25.0 | 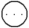 | 5000 |
| | | 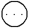 | 125 | 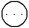 | 12.5 | 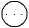 | 1250 | 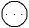 | 125 | 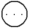 | 12.5 | 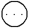 | 2500 |
| | | 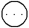 | 62.5 | 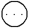 | 6.25 | 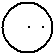 | 625 | | 62.5 | 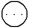 | 6.25 | 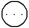 | 1250 |
| | | | 31.3 | 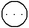 | 3.13 | | 313 | | 31.3 | 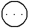 | 3.13 | 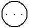 | 625 |
| | | | 15.6 | 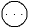 | 1.56 | | 156 | | 15.6 | | 1.56 | | 313 |
| | | | 7.8 | | 0.78 | | 78 | | 7.8 | | 0.78 | | 156 |
| LL | 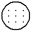 | | 0 | | 0 | | 0 | | 0 | | 0 | 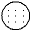 | LL |

A plate 57 ELF, 1 hr
Antigen Standard Curve Dilutions:
IL-4 = 500 to 7.8 pg/well
IL-8 = 50 to 0 78 pg/well
IL-10 = 5000 to 78 pg/well
DetectionAb Mixture
anti IL-4, 8, 10
1.100 dilution
diluent = 1 mg/mL casein in TBS
alternate columns contain diluent only
LL = landing lights, Hu IgG

As can be seen from Table 2, good sensitivity, linearity, and dynamic range were achieved with serial dilution of the antigens. In this example, IL-4 was detected from 7.8 pg/well to 250 pg/well (r²=0.98); IL-8 was detected from 0.8 pg/well to 50 pg/well; and IL-10 was detected from 31 pg/well to 500 pg/well (r²=0.97).

Unlike conventional plate assays, which have one capture molecule in each well, the system of the present invention uses plates which have a plurality of capture molecules in each well. Each capture molecule binds to the epitope of an analyte. In the case of antigens, each capture antibody binds to an epitope of the desired antigen. The match occurs with an antibody that recognizes different epitopes which do not cross-react with any other antibody or epitope.

To select antibodies for use in the present assay, which antibodies lack cross-reactivity, a capture antibody-antigen pair is challenged with incurred (labeled) secondary antibodies. Alternatively, the capture antibody is challenged with an incorrect antigen and a corresponding labeled secondary antibody. Once the antibodies are selected, all of the antigens are mixed together and the array is challenged with a single secondary antibody and then with (n+1) progressive mixtures of secondary antibodies. Thus, all cross-reactive issues are resolved by the array.

The system of the present invention uses an optimal abount of capture molecule for analyte and control in each spot to be analyzed. The concentration of antibody in the printing ink is varied, and then it is determined which concentration provides maximal antigen binding. This can be effected readily by one skilled in the art without undue experimentation. For various monoclonal antibodies, input (or loading) concentration in the range of about 0.25 mg/mL or about 1 mg/mL provide optimal binding and print down from about 200 pL to about 10 nL, depending upon the printer used. As can be appreciated by one skilled in the art, there are variations because of different Kd=s and activity among antibodies, as well as size and structure differences among antigens, which lead to steric hindrance.

The spots in the wells are on the order of about 100 microns or less. This system is possible because there is now equipment available to print the capture molecules in the desired density and size of spot in the wells of the plates without interfering with the other assays in the well. Using a strong light source, one can view many different assays at once in one sample or in a plurality of samples.

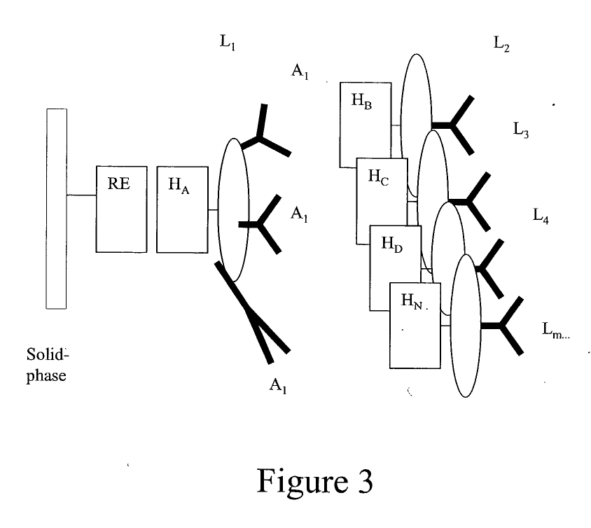

In another embodiment of the invention, arrays of molecular recognition elements (RE) are immobilized in the bottom of a multiple array plate wells in a pre-defined order such that their registration on both the x and y axes is known. In a non-limiting example, RE is an antibody that recognizes a specific hapten molecule, H. The hapten (HA) might be the dye, FITC, and the molecular recognition elements (REA) might be an anti-FITC immunoglobulin such that an immuno-affinity complex (REA(anti-FITC) . . . HA (FITC) is formed. The hapten can be conjugated to a carrier molecule such as albumin, or the hapten can be directly conjugated to a capture antibody. The hapten conjugate, illustrated in FIG. 3 as L, relates to the analyte (A) specific ligand. For example, L1 may be a capture antibody for the analyte A1. If L1 represents an FITCV conjugated bovine serum albumin that is also conjugated with an antibody that recognizes analyte A1, if A1 is interleukin 8, the L1 is capable of binding A1 to form a complex in solution as L(FITC)-BSA-(anti-IL8) . . . A(IL8). This complex in turn may be captured by >RE (anti-FITC) immobilized to the solid phase: REA(anti-FITC) . . . L(FITC)-BSA-(anti-IL8) . . . A(IL8).

To create the simplest form of a universal assay according to the present invention, the following reagents are required:

a library of anti-hapten antibodies; and

a library of corresponding haptenated capture antibodies.

The system of the present invention can be used for any type of immunoassay, including sandwich immunoassays, competitive immunoassays, ELISA, and the like. The capture molecules can be nucleic acids, proteins, or any capture molecule that binds exclusively with one other type of molecule.

The signal produced by an array may be detected by the naked eye or by means of a specifically designed instrument, such as a confocal array reader. In one embodiment, a fluorescent signal is recorded with a CCD camera. It will be appreciated by those skilled in the art that the choice of a particular method used to detect and quantify the signal is not crucial for the present invention. Thus, any detection method may be used as long as it provides consistent and accurate results.

The assay of the present invention provides controls for each individual assay, taking into consideration well to well and microarray to microarray variation issues. Each member of the array contains capture agents. In the case in which each member includes a control, then each member of the array contains, both analyte-specific capture (binding ) agents and control-specific capture agents. When each member does not include a control, then each member contains either an analyte-specific capture agent or a control-specific capture agent. The assays are conducted by a non-competitive method.

As noted above, the controls can be other proteins, haptens, labels, etc., so long as they are not members of the binding pair which includes the analyte capture probe. For example, if the analyte to be determined is TSH, then the analyte-specific capture agent is anti-TSH. To the TSH sample is introduced a control of known concentration. The control can be, for example, streptavidin-alkaline phosphatase conjugate, and the control specific capture agent can be biotin. The amount of biotin immobilized as a probe to the surface can be varied to create a series of capture spots that are capable of binding different amounts of streptavidin-alkaline phosphatase, resulting in different signal intensities, thereby establishing a control standard curve for biotin-streptavidin binding. The TSH antigen is detected by a sandwich assay in which a secondary antibody to TSH is binding labeled. Thus, introducing streptavidin alkaline phosphatase conjugate detects the presence of TSH and simultaneously sets up a biotin standard curve within each well. The enzyme conjugate distributes (competes) between the secondary antibody and the biotin control probes, and makes it possible to calculate the TSH antigen concentration.

Another embodiment of the assay of the present invention is when the labeled secondary antibody also contains a hapten. In this case, a hapten-specific binding agent or probe is immobilized along with the analyte-specific probes. The sample analyte is incubated, and unbound analyte is removed. Analyte-specific detection agent (e.g., the haptenated secondary antibody) is added. There is competitive binding of this reporter between analyte and hapten binding probe. The signal distributes between antibody and hapten capture probes, and can be used to determine analyte concentration.

In a similar example an analog is introduced that competes with analyte for the capture antibody binding. However, the secondary antibody is directed toward the analog and not the analyte. The secondary reporter can also be haptenated, and the distribution of signal between analog and hapten binding us used to determine analyte concentration in this competitive binding method.

In all cases, the microarrays contain both analyte-specific binding agents (e.g., capture antibody) and control-specific binding agents, either in the same or different spots or wells. Each member of the microarray or well is used to generate a standard curve. When using a plate having 96 wells, 96 different standard curves are generated.

The foregoing description of the specific embodiments will so fully reveal the general nature of the invention that other can, by applying current knowledge, readily modify and/or adapt for various application such specific embodiments without undue experimentation and without departing from the generic concept. Therefore, such adaptations and modifications should and are intended to be comprehended within the meaning and range of equivalents of the disclosed embodiments.

It is to be understood that the phraseology or terminology employed herein is for the purpose of description and not of limitation. The means and materials for carrying out various disclosed functions may take a variety of alternative forms without departing from the invention.

Thus, the expressions Ameans to@ and Ameans for@ as may be found in the specification above and/or in the claims below, followed by a functional statement, are intended to define and cover whatever structural, physical, chemical, or electrical element or structures which may now or in the future exist for carrying out the recited function, whether or nor precisely equivalent to the embodiment or embodiments disclosed in the specification above. It is intended that such expressions be given their broadest interpretation.

All references cited herein are incorporated by reference.

What is claimed is:

1. A method for assaying multiple analytes comprising:
a. contacting a surface of a substrate with an array, wherein at least one member of the array comprises a capture element for a specific analyte, a capture element for a control or a combination of a capture element for a specific analyte and a capture element for a control;
b. delivering an analyte, a control, or an analyte and a control, to at least one member of the array on the surface and incubating the analytes and controls to form complexes;
c. rinsing the surface to remove unbound analytes and controls;
d. delivering to at least one member of the array a solution of the same or different molecular recognition signal reporters that bind to the analytes and to the controls;
e. incubating the molecular recognition signal reporters, and rinsing to remove unbound molecular recognition signal reporters;
f. adding signal generation reagents;
g. detecting the signals generated from bound analytes and from bound controls.

2. The method according to claim 1 wherein the substrate is a multi-well plate.

3. The method according to claim 1 wherein each member of the array includes both a capture element for a specific analyte and a capture element for a control.

4. The method according to claim 1 wherein each member of the array includes either a capture element for a specific analyte or a capture element for a control.

5. The method according to claim 1 wherein the analytes and controls are delivered to the substrate randomly, sequentially, or in parallel to the substrate.

6. The method according to claim 1 wherein the signal generation reagents are all the same.

7. The method according to claim 1 wherein the signal generation reagents are different for each analyte or control.

8. The method according to claim 1 wherein the capture elements, analytes, and controls are selected from the group consisting of proteins, peptides, aptamers, antibodies, antigens, enzymes, haptens, and receptors, and analogs and mimics thereof.

9. The method according to claim 8 wherein the antigens are selected from the group consisting of interleukins, cytokines, chemokines, growth factors, hormones, and transcription factors.

10. The method according to claim 8 wherein the capture elements, analytes, and controls are selected from proteins associated with cytokine signaling pathways, MAP kinases, Akt signaling pathways, PKC pathways, apoptosis and capsase signaling pathways, and proteins involved in cell cycle and translational control.

11. The method according to claim 8 wherein the capture elements, analytes, and controls are selected from proteins that participate in or are associated with phosphorylation, dephosphorylation, glycosylation, deglycosylation, acylation, deacylation, methylation, or demethylation of molecules.

12. The method according to claim 1 wherein the molecular recognition signal reporters are selected from the group consisting of labeled forms of proteins, peptides, aptamers, antibodies, antigens, enzymes, haptens, and receptors, and analogs and mimics thereof.

13. The method according to claim 12 wherein the molecular recognition signal reporters are selected from the group consisting of enzymes, enzyme substrates, stable isotope mass tags, labels for mass spectroscopy, radiolabels, and hapten conjugates or complexes of said molecular recognition signal reporters.

14. The method according to claim 1 wherein the controls are selected from the group consisting of antigens and analogs and mimics thereof.

15. The method according to claim 1 wherein the capture elements comprise haptenated proteins conjugated with capture antibodies, or analogs or mimics thereof.

16. The method according to claim 15 wherein the array is formed by self-assembly of the capture elements onto corresponding anti-hapten antibodies, aptamers, and analogs or mimics thereof which are arrayed on the surface of the substrate at defined locations.

17. The method according to claim 16 wherein the haptenated proteins are selected from the group consisting of interleukins, cytokines, chemokines, growth factors, hormones, and transcription factors.

18. The method according to claim 1 wherein the capture elements, analytes, and controls are selected from the group consisting of proteins associated with cytokine signaling pathways, MAP kinases, Akt signaling pathways, PKC pathways, apoptosis and caspase signaling pathways, and proteins involved in cell cycle and translational control.

19. The method according to claim 8 wherein the molecular recognition reporters are selected from labeled members of the group consisting of proteins, peptides, aptamers, antibodies, antigens, enzymes, haptens, and receptors, and analogs and mimics thereof.

20. The method according to claim 1 wherein the capture elements, analytes, and controls are selected from proteins that participate in or are associated with phosphorylation, dephosphorylation, glycosylation, deglycosylation, acylation, deacylation, methylation, and demethylation of molecules.

21. The method according to claim 1 wherein the molecular recognition reporters are selected from the group consisting of enzymes, enzyme substrates, dyes, metal, radiolabels, and hapten conjugates or complexes of the molecular recognition reporters.

22. The method according to claim 1 wherein the array of capture elements comprises antibodies, their analogs, and mimics thereof, and the controls comprise antigens, their analogs, and mimics thereof.

23. The method according to claim 1 wherein the capture elements are selected from the group consisting of haptenated proteins conjugated with capture antibodies, aptamers, and analogs or mimics thereof.

24. The method according to claim 2 wherein the multi-well plate has an activated surface.

25. The method according to claim 24 wherein the surface of the multi-well plate is activated with at least one acyl fluoride group.

26. The method according to claim 25 wherein the activated surface comprises nucleophilic, electrophilic, photoreactive, or metal binding groups.

27. The method according to claim 1 wherein the assay is conducted by a computer-controlled automated device.

28. The method according to claim 27 wherein the computer-controlled automated device is a robotic device.

29. The method according to claim 1 wherein analytes are added to successive wells in serial dilution to measure limits of detection and dynamic range.

30. A kit for assaying multiple analytes comprising:
a. a multiwell plate comprising an array of members, wherein each member comprises a capture element for a specific analyte, a capture element for a control, or a combination of a capture element for a specific analyte and a capture element for a control;
b. molecular recognition agents for specific analytes;
c. molecular recognition agents for controls;
d. signal reporters; and
e. signal generation reagents.

31. The kit according to claim 30 wherein each member comprises both a capture agent for a specific analyte and a capture agent for a control.

32. The kit according to claim 30 wherein each member comprises a capture agent for a specific analyte or a capture agent for a control.

33. The kit according to claim 30 wherein the multiwell plate has an activated surface.

34. The kit according to claim 33 wherein the surface of the multiwell plate is activated with at least one acyl fluoride group.

35. The kit according to claim 33 wherein the surface of the multiwell plate is activated with nucleophilic, electrophilic, photoreactive, or metal binding groups.
